(19) United States
(12) Patent Application Publication
(10) Pub. No.: US 2025/0073335 A1
(43) Pub. Date: Mar. 6, 2025

(54) PHARMACEUTICAL COMPOSITION CONTAINING CHIRAL NANOZYME AND METHOD OF PREVENTING OR TREATING PROLIFERATIVE DISEASES USING THE SAME

(71) Applicants: EWHA UNIVERSITY-INDUSTRY COLLABORATION FOUNDATION, Seoul (KR); SEOUL NATIONAL UNIVERSITY R&DB FOUNDATION, Seoul (KR)

(72) Inventors: Dong-Ha KIM, Seoul (KR); Su-Bin YU, Seoul (KR); Ha-Eun KANG, Seoul (KR); Ki-Tae NAM, Seoul (KR); Ryeong-Myeong KIM, Gumi-si (KR)

(21) Appl. No.: 18/950,312

(22) Filed: Nov. 18, 2024

Related U.S. Application Data
(63) Continuation of application No. PCT/KR2023/007066, filed on May 24, 2023.

(30) Foreign Application Priority Data
May 24, 2022 (KR) 10-2022-0063450

(51) Int. Cl. A61K 41/00

(52) U.S. Cl. CPC A61K 41/0057; A61K 41/0052

(57) ABSTRACT

The present disclosure relates to a pharmaceutical composition comprising chiral nanozyme and method of preventing or treating proliferative diseases using the same. A first nanozyme and/or a second nanozyme according to embodiments of the present disclosure has a chiral structure, and their enzymatic activity is maximized when circularly polarized light with the same directionality as the first nanozyme and/or the second nanozyme is irradiated.

### CROSS-REFERENCE TO RELATED APPLICATION

This application is a continuation of PCT Application No. PCT/KR2023/007066, filed on May 24, 2023, which claims priority to Korean Patent Application Number 10-2022-0063450, filed on May 24, 2022, both of which are hereby incorporated by reference in their entirety.

### TECHNICAL FIELD

The present disclosure provides a pharmaceutical composition including chiral nanozyme and method of preventing or treating proliferative diseases using the same.

### BACKGROUND

Nanozymes are substances that exhibit enzyme-like activities which are characteristics of noble metals, such as Au and Ag, and transition metals, such as Pd, Pt, and Mn. The reactions mimicked by nanozymes are mostly oxidation and reduction reactions, and it has been reported that they mimic the activities of enzymes, such as peroxidase, oxidase, catalase, and superoxide dismutase. In addition, nanoparticles exhibiting the activities of esterase, nuclease, phosphatase, and protease have also been reported. It is known that the activities of nanozymes arise from the catalytic activities of elements that make up the particles, and the magnitudes and characteristics of the activities can vary greatly depending on the shape, size, structure, and surface state of the nanoparticles. Thus, it is possible to control the activities of nanozymes to some extent according to the intended application.

Inorganic-based nanozymes exhibit significantly stable activities against external environmental factors, such as pH and temperature, and can be chemically synthesized in large quantities at 1000 times lower cost compared to organic enzymes. Therefore, the inorganic-based nanozymes are gaining attention as a new paradigm of artificial enzymes that can replace conventional protein enzymes.

However, nanozymes are nanostructures that do not inherently have a characteristic active site and thus lack substrate selectivity. Also, inorganic nanozymes generally have lower activities than organic enzymes yet. Therefore, recent research has focused on the development of new nanozymes that can overcome such disadvantages, and further research is being carried out to verify clinical utility for actual commercialization.

### PRIOR ART LITERATURE

Korean Patent Registered Publication No. 10-2081666.

### DISCLOSURE OF THE INVENTION

### Problems to be Solved by the Invention

The present disclosure provides a pharmaceutical composition including chiral nanozyme and method of preventing or treating proliferative diseases using the same.

However, problems to be solved by the present disclosure are not limited to the above-described problems. Although not described herein, other problems to be solved by the present disclosure can be clearly understood by a person with ordinary skill in the art from the following description.

### Means for Solving the Problems

A first aspect of the present disclosure provides a first nanozyme pharmaceutical composition including: a first nanozyme including a first metal nanostructure having a chiral structure, and the first nanozyme's enzymatic activity is improved when circularly polarized light is irradiated thereto and the first nanozyme pharmaceutical composition is used for preventing or treating proliferative diseases.

A second aspect of the present disclosure provides a second nanozyme pharmaceutical composition including: a second nanozyme including a second metal nanostructure having a chiral structure and a metal coating layer, and the second nanozyme's enzymatic activity is improved when circularly polarized light is irradiated thereto, and the second nanozyme pharmaceutical composition is used for preventing or treating proliferative diseases.

A third aspect of the present disclosure provides a method of preventing or treating proliferative diseases, including: a process of simultaneously or sequentially injecting a first nanozyme pharmaceutical composition of the first aspect and a second nanozyme pharmaceutical composition of the second aspect into non-human subjects; and a process of irradiating right-handed circularly polarized light and left-handed circularly polarized light in sequence to each of respective sites injected with the first nanozyme pharmaceutical composition and the second nanozyme pharmaceutical composition.

### Effects of the Inventions

A first nanozyme and/or a second nanozyme according to embodiments of the present disclosure has a chiral structure, and their enzymatic activity is maximized when circularly polarized light with the same directionality as the first nanozyme and/or the second nanozyme is irradiated.

The first nanozyme and/or the second nanozyme according to embodiments of the present disclosure has the same directionality as the substrate and thus has improved substrate selectivity. For example, a chiral nanozyme containing gold (Au) with D-directionality has the same directionality as D-glucose in the human body and thus has improved substrate interactions, thereby increasing the glucose oxidase-like activity of Au.

A first nanozyme pharmaceutical composition and a second nanozyme pharmaceutical composition according to embodiments of the present disclosure can be simultaneously or sequentially injected into non-human subjects, and when right-handed circularly polarized light and left-handed circularly polarized light are irradiated in sequence to each of the respective sites injected with the first nanozyme pharmaceutical composition and the second nanozyme pharmaceutical composition, a continuous enzymatic reaction occurs, D-glucose is used as a substrate to finally produce hydroxyl radicals (·OH) through consecutive enzymatic reactions. The produced hydroxyl radicals (·OH) attack cancer cells, and thus, it is possible to prevent or treat cancer. Also, the first nanozyme pharmaceutical composition and/or the second nanozyme pharmaceutical composition according to embodiments of the present disclosure can reduce the amount of glucose, which is a nutrient source for cancer cells, from cancer cells through glucose oxidase-like activity reactions and thus has a starvation therapy effect.

The first nanozyme pharmaceutical composition and/or the second nanozyme pharmaceutical composition according to embodiments of the present disclosure can have a photothermal therapy effect and/or a photodynamic therapy effect due to the plasmonic properties of inorganic metals.

### BRIEF DESCRIPTION OF THE DRAWINGS

FIG. 1 is a schematic diagram illustrating consecutive enzymatic reactions of a first nanozyme pharmaceutical composition and a second nanozyme pharmaceutical composition according to an embodiment of the present disclosure.

FIG. 2 is a schematic diagram illustrating a process of producing an L-Au nano particle having a Pd coating layer (L-Au NP@Pd) according to an example of the present disclosure.

FIG. 3A and FIG. 3B are scanning electron microscope (SEM) images of L-Au NP according to an example of the present disclosure.

FIG. 4A and FIG. 4B are SEM images of L-Au NP@Pd according to an example of the present disclosure.

FIG. 5A to FIG. 5C are an SEM image of L-Au NP@Pd, an energy dispersive x-ray (EDX) image of Pd, and an EDX image of Au, respectively, according to an example of the present disclosure.

FIG. 6 shows an absorption spectrum of L-Au NP@Pd according to an example of the present disclosure.

FIG. 7 shows a circular dichroism (CD) spectrum of L-Au NP@Pd according to an example of the present disclosure.

FIG. 8 shows tetramethylbenzidine (TMB) absorption spectra of L-Au NP@Pd in the presence of hydrogen peroxide and under irradiation of an 808 nm laser according to an example of the present disclosure.

FIG. 9 shows TMB absorption spectra of L-Au NP@Pd under right-handed circularly polarized light (RCP), left-handed circularly polarized light (LCP), and linearly polarized light (LP) according to an example of the present disclosure.

FIG. 10 shows Amplex Red fluorescence spectra of D-Au NP under LCP, LP, and RCP according to an example of the present disclosure.

FIG. 11 and FIG. 12 show fluorescence spectra derived by Amplex Red assay of D-Au NP and L-Au NP, respectively, according to an example of the present disclosure.

FIG. 13 shows terephthalic acid (TA) fluorescence spectra after treating samples containing D-Au NP and L-Au NP@Pd, both with and without D-glucose, according to an example of the present disclosure.

FIG. 14 shows a dark in vitro cytotoxicity graph of D-Au NP (D-Au NP@PEG) and L-Au NP@Pd (L-Au NP@Pd@PEG) treated with polyethylene glycol (PEG) according to an example of the present disclosure.

FIG. 15 shows in vitro cytotoxicity graph of D-Au NP@PEG, L-Au NP@Pd@PEG, and D-Au NP@PEG+L-Au NP@Pd@PEG under irradiation of light according to an example of the present disclosure.

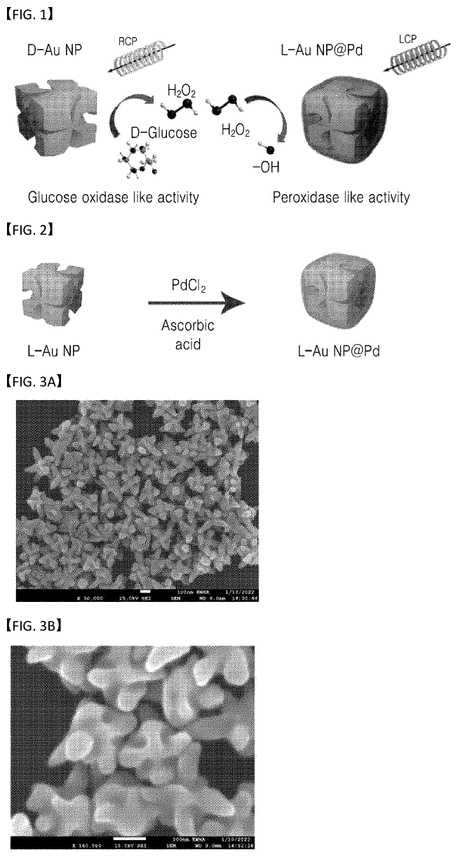

### BEST MODE FOR CARRYING OUT THE INVENTION

Hereafter, embodiments of the present disclosure will be described in detail with reference to the accompanying drawings. However, it is to be noted that the present disclosure is not limited to the embodiments but can be embodied in various other ways. Also, the accompanying drawings are provided to help easily understand the embodiments of the present disclosure and the technical conception described in the present disclosure is not limited by the accompanying drawings. In the drawings, parts irrelevant to the description are omitted for the simplicity of explanation, and like reference numerals denote like parts through the whole document.

Through the whole document, the term “connected to” or “coupled to” that is used to designate a connection or coupling of one element to another element includes both a case that an element is “directly connected or coupled to” another element and a case that an element is “electronically connected or coupled to” another element via still another element.

Through the whole document, the term “on” that is used to designate a position of one element with respect to another element includes both a case that the one element is adjacent to the other element and a case that any other element exists between these two elements.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise.

Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party.

Through the whole document, the term “step of” does not mean “step for”.

Through the whole document, the term “combination of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Through this whole specification, a phrase in the form “A and/or B” means “A or B, or A and B”.

Hereinafter, embodiments and examples of the present disclosure will be described in detail with reference to the accompanying drawings. However, the present disclosure may not be limited to the following embodiments, examples, and drawings.

A first aspect of the present disclosure provides a first nanozyme pharmaceutical composition including a first nanozyme including a first metal nanostructure having a chiral structure, and the first nanozyme's enzymatic activity is improved when circularly polarized light is irradiated thereto and the first nanozyme pharmaceutical composition is used for preventing or treating proliferative diseases.

In an embodiment of the present disclosure, the first metal nanostructure may have D-directionality or L-directionality, and the first nanozyme may have D-directionality or L-directionality depending on the directionality of the first metal nanostructure.

In an embodiment of the present disclosure, the circularly polarized light may be left-handed circularly polarized light or right-handed circularly polarized light.

In an embodiment of the present disclosure, the first metal nanostructure may include at least one selected from Au, Pd, Pt, Ce, Mn, Ir, and Rh, but may not be limited thereto.

In the embodiment of the present disclosure, the first nanozyme may exhibit glucose oxidase-like activity.

In an embodiment of the present disclosure, the glucose oxidation reaction may reduce glucose, which is a nutrient source for cancer cells, from cancer cells to induce starvation therapy.

In an embodiment of the present disclosure, the first nanozyme may have D-directionality, but is not limited thereto. In an embodiment of the present disclosure, the first nanozyme may have D-directionality since the first metal nanostructure has D-directionality. In an embodiment of the present disclosure, the first nanozyme may produce H₂O₂ by using D-glucose as a substrate.

A second aspect of the present disclosure provides a second nanozyme pharmaceutical composition including a second nanozyme including: a second metal nanostructure having a chiral structure; and a metal coating layer, and the second nanozyme's enzymatic activity is improved when circularly polarized light is irradiated thereto and is used for preventing or treating proliferative diseases.

Detailed descriptions of the second aspect of the present disclosure, which overlap with those of the first aspect of the present disclosure, are omitted hereinafter, but the descriptions of the first aspect of the present disclosure may be identically applied to the second aspect of the present disclosure, even though they are omitted hereinafter.

In an embodiment of the present disclosure, the second metal nanostructure may have D-directionality or L-directionality, and the second nanozyme may have D-directionality or L-directionality depending on the directionality of the second metal nanostructure.

In an embodiment of the present disclosure, the circularly polarized light may be left-handed circularly polarized light or right-handed circularly polarized light.

In an embodiment of the present disclosure, the second metal nanostructure may include at least one selected from Au, Pd, Ce, Fe, Cu, Co and Ir, but may not be limited thereto.

In an embodiment of the present disclosure, the metal coating layer may comprise at least one selected from Au, Pd, Pt, Ce, Fe, Cu, Mn, Mo, Co, Ir, Ru and Ag, but may not be limited thereto.

In an embodiment of the present disclosure, the second metal nanostructure and the metal coating layer may contain the same metal or different metals.

In an embodiment of the present disclosure, the second nanozyme may exhibit peroxidase-like activity. In an embodiment of the present disclosure, the second nanozyme may produce hydroxyl radicals (·OH) by using H₂O₂ as a substrate.

In an embodiment of the present disclosure, the second nanozyme may have L-directionality. in an embodiment of the present disclosure, the second nanozyme may have L-directionality depending on L-directionality of the second metal nanostructure.

In an embodiment of the present disclosure, properties of the second nanozyme may be determined by the metal coating layer.

A third aspect of the present disclosure provides a method of preventing or treating proliferative diseases, including a process of simultaneously or sequentially injecting a first nanozyme pharmaceutical composition according to the first aspect and a second nanozyme pharmaceutical composition according to the second aspect into non-human subjects; and a process of irradiating right-handed circularly polarized light and left-handed circularly polarized light in sequence to each of respective sites injected with the first nanozyme pharmaceutical composition and the second nanozyme pharmaceutical composition.

Detailed descriptions of the third aspect of the present disclosure, which overlap with those of the first and the second aspect of the present disclosure, are omitted hereinafter, but the descriptions of the first and the second aspect of the present disclosure may be identically applied to the third aspect of the present disclosure, even though they are omitted hereinafter.

In an embodiment of the present disclosure, hydroxyl radicals may be produced from D-glucose by the above-described method.

In an embodiment of the present disclosure, each of the first metal nanostructure and the second metal nanostructure may contain Au, and the metal coating layer of the second nanozyme may contain Pd. The first nanozyme may have a chiral structure with D-directionality, and the second nanozyme may have a chiral structure with L-directionality. In this case, when right-handed circularly polarized light is irradiated to the first nanozyme, H₂O₂ may be produced by using D-glucose as a substrate through glucose oxidase-like activity of Au, and when left-handed circularly polarized light is irradiated to the second nanozyme, H₂O₂ may be oxidized and hydroxyl radicals (·OH) may be produced through peroxidase-like activity of Pd. This can be used for photodynamic therapy.

In an embodiment of the present disclosure, the method of preventing or treating proliferative diseases may include injecting the first nanozyme pharmaceutical composition and the second pharmaceutical composition into human or non-human subjects, but may not be limited thereto. Also, in an embodiment of the present disclosure, the method of preventing or treating proliferative diseases may include injecting the first nanozyme pharmaceutical composition and the second pharmaceutical composition into non-human subjects.

In an embodiment of the present disclosure, the pharmaceutical composition may be administered to humans or non-human subjects by any applicable administration method without limitations.

In an embodiment of the present disclosure, the method of preventing or treating proliferative diseases may have a photothermal therapy effect and/or a photodynamic therapy effect. In an embodiment of the present disclosure, the method of preventing or treating proliferative diseases may have a photothermal therapy effect due to the plasmonic properties of inorganic metals by having the first metal nanostructure or the second metal nanostructure.

In an embodiment of the present disclosure, the proliferative diseases may include at least one selected from solid tumor, blood cancer, colorectal cancer, uterine cancer, uterine fibroid, meningioma, lung cancer, small cell lung cancer, gastrointestinal cancer, colon cancer, intestinal cancer, breast cancer, ovarian cancer, prostate cancer, testicular cancer, liver cancer, kidney cancer, bladder cancer, pancreatic cancer, brain cancer, sarcoma, osteosarcoma, Kaposi's sarcoma, and melanoma.

In an embodiment of the present disclosure, the method of preventing or treating proliferative diseases may produce a large amount of active oxygen species from glucose around cancer lesions through consecutive enzymatic reactions.

Hereinafter, the present disclosure will be explained in more detail with reference to Examples. However, the following Examples are illustrated only for better understanding of the present disclosure but do not limit the present disclosure.

### MODE FOR CARRYING OUT THE INVENTION

### EXAMPLES

### Example 1. Synthesis of D-Au Nanoparticle (D-Au NP)

0.8 mL of 100 mM hexadecyltrimethylammonium bromide (CTAB) was added to 3.95 mL of water. In this solution, 0.1 mL of 10 mM gold precursor tetrachloroauric (III) trihydrate (HAuCl₄·3H₂O) and 0.475 mL of 0.1 M L-ascorbic acid, a reducing agent, were mixed and stirred with a vortex mixer for 1 minute to prepare a first mixed solution. Then, 0.5 μL of 1 mM D-glutathione dissolved in water was added to the first mixed solution and stirred with a vortex mixer for 1 minute to prepare a second mixed solution. Octahedral or cubic Au nanoparticles with a size of 45 nm were added to the second mixed solution, and after 2 hours, a metal nanostructure having a chiral structure controlled by D-glutathione was synthesized. After that, the obtained metal nanostructure was then washed and separated by centrifugation at 5000 rpm for 30 seconds.

### Example 2. Synthesis of L-Au Nanoparticle Having Pd Coating Layer (L-Au NP@Pd)

### 2-1. Synthesis of L-Au Nanoparticle (L-Au NP)

Au nanoparticles having an L-chiral structure (L-Au NP) were synthesized by the same preparation method as in Example 1 except that L-glutathione was added as a peptide.

### 2-2. Synthesis of L-Au NP@Pd (see FIG. 2)

2 mL of L-Au NP (Optical Density (OD): 2.0) synthesized in Example 2-1 was dispersed in hexadecyltrimethylammonium chloride (CTAC) and then mixed and stirred with 10 mM PdCl₂ and 10 mM ascorbic acid, a reducing agent, followed by incubation for one day. Subsequently, after removal of the remaining PdCl₂ by centrifugation, redispersion was carried out in CTAB.

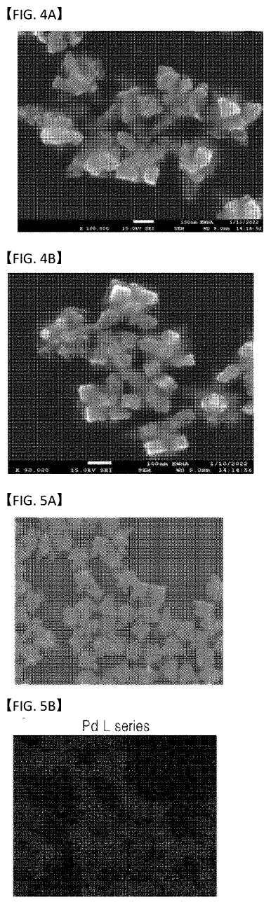

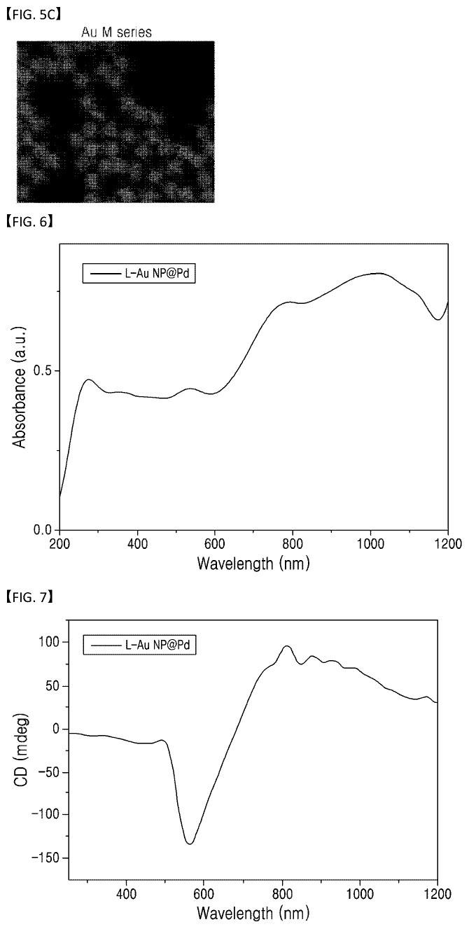

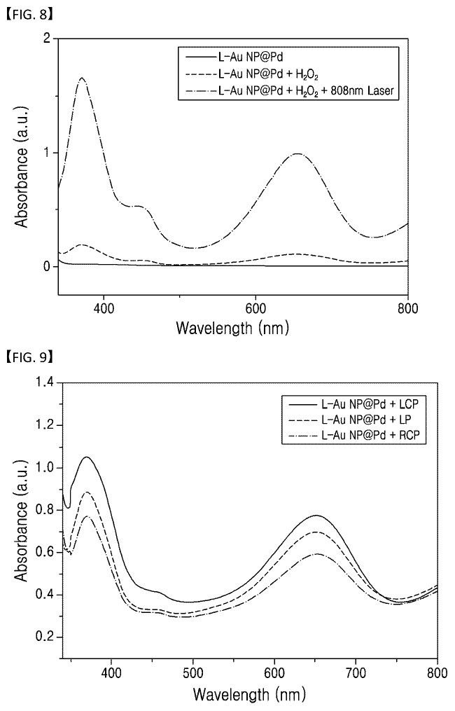

FIG. 3A and FIG. 3B are scanning electron microscope (SEM) images of L-Au NP, and FIG. 4A and FIG. 4B are SEM images of L-Au NP with Pd on the surface (L-Au NP@Pd). It can be seen that L-Au NP shows a smooth surface, whereas L-Au NP@Pd exhibits bumpy surfaces, demonstrating the successful deposition of the Pd NP shell on the L-Au surface. Also, FIG. 5A to FIG. 5C show energy dispersive X-ray (EDX) analysis data of L-Au NP@Pd, which confirms the introduction of Pd.

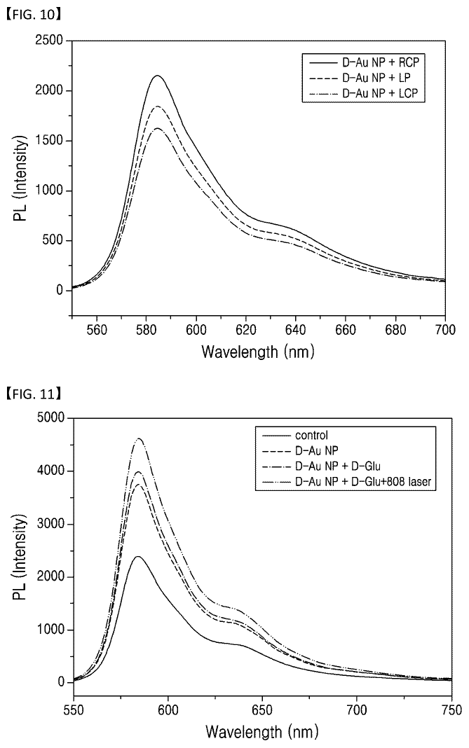

Also, it can be seen from the absorption spectrum in FIG. 6 that L-Au NP@Pd shows absorption in the near-infrared region of 800 nm to 1000 nm, and it can be seen from the circular dichroism (CD) spectrum in FIG. 7 that in the near-infrared region (808 nm), L-Au NP@Pd absorbs left-handed circularly polarized light (LCP) more strongly than right-handed circularly polarized light (RCP).

### Example 3. Preparation of Chiral Hybrid Nanozyme Composition Containing D-Au NP Nanoparticle and L-Au NP@Pd Nanoparticle

A chiral hybrid nanozyme composition containing D-Au NP prepared as in Example 1 and L-Au NP@Pd prepared as in Example 2 was prepared.

### Test Example 1. Enzymatic Activity Assay of L-Au NP@Pd and D-Au NP

### 1-1. Peroxidase-Like Activity Assay of L-Au NP@Pd Through 3,3′,5,5′-tetramethylbenzidine (TMB) Test

To confirm the peroxidase-like activity of L-Au NP@Pd, the absorption spectra were checked after allowing L-Au NP@Pd to react with 3,3′,5,5′-tetramethylbenzidine (TMB).

### 1) Test Procedure

A 0.1 M acetate buffer solution was prepared by dissolving sodium acetate in distilled water, and the pH was adjusted to 4.5 by using acetic acid. Then, 20 μL of 0.12 M tetramethylbenzidine (TMB) dissolved in dimethyl sulfoxide (DMSO), 200 μL of L-Au NP@Pd, and 8 μL of 30% H₂O₂ were added to the acetate buffer solution, and a reaction was conducted under LCP, LP, and RCP laser. The reaction was conducted at 35° C. to 40° C., after 30 minutes of reaction, UV-vis absorption was measured.

TMB reacts with a peroxidase substrate and is transformed into oxidized TMB (ox-TMB), which is blue in color, and it shows a peak at about 650 nm in an absorption graph.

### 2) Result

Referring to FIG. 8, it can be seen that L-Au NP@Pd exhibits peroxidase-like activity when an 808 nm laser is irradiated in the presence of hydrogen peroxide.

FIG. 9 shows the levels of enzymatic activity depending on the polarization direction of light irradiated onto L-Au NP@Pd. L-Au NP@Pd showed the highest enzymatic activity under LCP, followed by LP and RCP. This corresponds to the result as described above with reference to FIG. 7 that absorption is greater under LCP than RCP in the near-infrared region of the CD (circular dichroism) spectrum of L-Au NP@Pd.

### 1-2. Glucose Oxidase-Like Activity Assay of D-Au NP Through Amplex Red Assay Test

To confirm the glucose oxidase like activity of D-Au NP, Amplex Red assay was conducted.

### 1) Preparation of Working Solution

A working solution was prepared by adding 485 μL of a reaction buffer solution, 5 μL of an Amplex Red reagent, and 10 μL of HRP to an Amplex® Red Hydrogen Peroxide/Peroxidase Assay Kit (Thermo Fisher Scientific Inc.) with stirring. Amplex Red is a reagent that can detect hydrogen peroxide and can show a fluorescence peak at about 570 nm when reacting with hydrogen peroxide.

### 2) Reaction Condition

A sample was prepared by adding 200 μL of D-Au NP (OD: 2) and 200 μL of 0.2 M D-glucose to 1 mL of distilled water and allowing the mixture to react for 30 minutes under an 808 nm laser. Also, three control samples were prepared: one with only distilled water, one with only D-Au NP, and one with a mixture of D-Au NP and D-glucose, a substrate, without laser irradiation. Tests were performed under the same conditions. After the four samples were allowed to react on a hot plate at 35° C. for 30 minutes, the supernatant was obtained by centrifugation. After 25 μL of the obtained supernatant and 25 μL of the working solution were mixed with 950 mL of distilled water, the mixture was incubated in an oven at 30° C. for 30 minutes and photoluminescence was measured.

### 3) Result

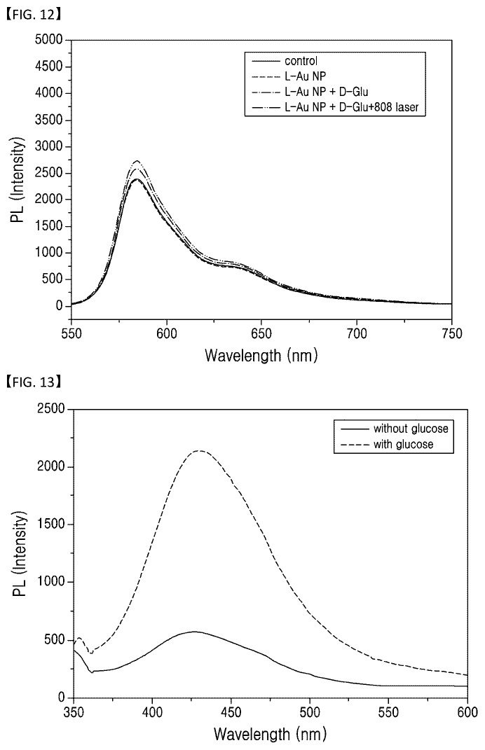

Referring to FIG. 10, it can be seen that D-Au NP showed the highest fluorescence under RCP, followed by LP and LCP. This indicates that the enzymatic activity was highest under RCP. This is the opposite trend from the activity of L-Au NP@Pd observed in the TMB test, which is attributed to the opposite chirality of gold nanoparticles.

### Test Example 2. Substrate Selectivity Assay of L-Au NP and D-Au NP

To confirm the effect of chiral nanozymes on substrate selectivity, each of D-Au NP and L-Au NP was evaluated for glucose oxidase-like activity using D-glucose as a substrate.

FIG. 11 and FIG. 12 show fluorescence spectra derived by Amplex Red assay of D-Au NP and L-Au NP, respectively. Referring to FIG. 11 and FIG. 12, each of D-Au NP and L-Au NP exhibited the highest absorption when D-glucose was added and an 808 nm laser was irradiated thereto, which confirms their glucose oxidase-like activity.

Comparing the enzymatic activity level of D-Au NP in FIG. 11 with the enzymatic activity level of L-Au NP in FIG. 12, it can be seen that the enzymatic activity is significantly higher in D-Au NP. This means that D-Au NP shows higher substrate selectivity for D-glucose, which confirms that enzymatic activity can be effectively increased by selecting a nanozyme with appropriate directionality for the substrate.

### Test Example 3. Assay on Production of Hydroxyl Radical by Chiral Hybrid Nanozyme Composition Containing D-Au NP and L-Au NP@Pd

To confirm that hydroxyl radicals are finally produced from D-glucose through consecutive enzymatic reactions of the hybrid nanozyme composition containing D-Au NP and L-Au NP@Pd, a test was conducted by using terephthalic acid (TA). TA is a reagent that detects hydroxyl radicals, and produces a strong fluorescence peak at about 450 nm when reacting with hydroxyl radicals.

### 1) Test Procedure

A 10 mM terephthalic acid (TA) solution was prepared by dissolving TA in 0.1 M NaOH. After 500 μL of D-Au NP (OD: 2) and 200 μL of 0.2 M D-glucose were stirred with 300 μL of distilled water, an RCP laser was irradiated for 15 minutes. 200 μL of L-Au NP@Pd (OD: 2) and 200 μL of the 10 mM TA solution were added to the solution, followed by irradiation of an LCP laser for 15 minutes. After the reaction was completed, the solution was centrifuged and 1 ml of the supernatant was collected to measure photoluminescence. A test was conducted on a sample containing only D-Au NP and L-Au NP@Pd without D-glucose as a control group.

### 2) Result

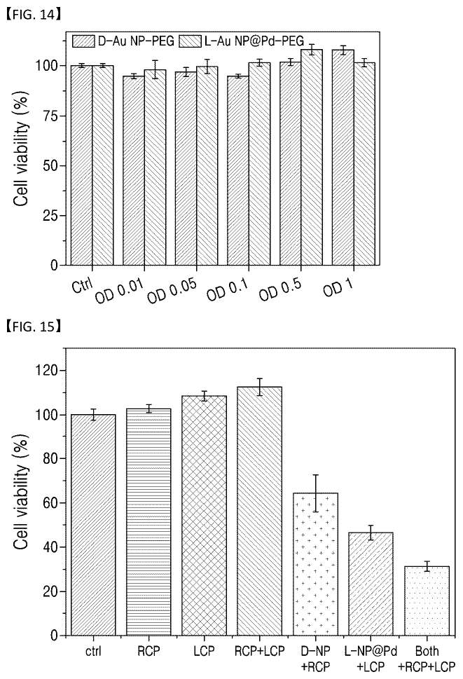

Referring to FIG. 13, a strong fluorescence peak was observed from the sample containing D-glucose, which confirms that hydroxyl radicals are effectively produced through cascade enzymatic activity using D-glucose as a substrate.

### Test Example 4. Cytotoxicity Assay (MTT Assay) Using Chiral Hybrid Nanozyme Composition Containing D-Au NP and L-Au NP@Pd

### In Vitro MTT Assay

After confirming the enzymatic activity of D-Au NP and L-Au NP@Pd through Test Examples 1 to 3, an in vitro test was conducted to check their toxicity within actual cells.

After D-Au NP and L-Au NP@Pd were treated with polyethylene glycol (PEG) and then applied to cells, cytotoxicity was measured via WST assay using Ez-cytox. Ez-cytox reacts with mitochondrial NADH-dehydrogenase in living cells to produce orange-colored soluble formazan, which has absorbance at 450 nm. This absorbance can be measured using a multi-reader to calculate formazan production, which correlates with the number of living cells. Thus, it is possible to measure toxicity. Hela cells were used as cancer cells, and Dulbecco's Modified Eagle's Media (DMEM) containing glucose was used as a cell medium.

### 4-1. Dark Toxicity Study

### 1) Test Procedure

To check toxicity in cells in the absence of laser irradiation, the following test was conducted. To enhance the biocompatibility of the materials, D-Au NP (OD: 1) and L-Au NP@Pd (OD: 1) were each prepared and redispersed in O-[2-(3-mercaptopropionylamino)ethyl]-O′-methylpolyethylene glycol (PEG-thiol) at 1 mg/mL. The solutions were incubated for a day and then redispersed in the cell media (DMEM) by centrifugation. PEG-introduced D-Au NP (D-Au NP@PEG) and L-Au NP@Pd (L-Au NP@Pd@PEG) were diluted to concentrations of OD 0.01, 0.05, 0.1, and 1 with DMEM and added to each well of a 96-well plate containing cultured cells. A sample group containing only D-Au NP@PEG, a sample group containing only L-Au NP@Pd@PEG, and a sample group containing both D-Au NP@PEG and L-Au NP@Pd@PEG were prepared, and a sample group without containing the above-described materials and a sample group without laser treatment were prepared as control groups to conduct a test. After allowing a day for the materials to enter the cells, Ez-cytox was diluted tenfold with DMEM and 100 μL of the diluted solution was added to each well and incubated for 30 minutes. Then, absorbance at 450 nm was measured by using a multi-reader.

### 2) Result

Dark toxicity in the absence of laser irradiation is shown in the graph of FIG. 14. Referring to FIG. 14, it can be seen that D-Au NP@PEG and L-Au NP@Pd@PEG do not exhibit significant toxicity in the cells in the range of OD 0.01 to OD 1.

### 4-2. Light Toxicity Study

### 1) Test Procedure

To check toxicity in cells under laser irradiation, the following test was conducted by using a circularly polarized laser (CPL). A process of introducing PEG and treating the cells in the 96-well plate was prepared in the same manner as described above in section 1) of 4-1. After the cells were treated with D-Au NP@PEG (OD: 1) and L-Au NP@Pd@PEG (OD: 1), the circularly polarized laser was irradiated thereto for 10 minutes, followed by treatment with Ez-cytox to measure toxicity. A sample without treatment with the materials and the laser was used as a control group. To check toxicity caused by the laser, a sample without treatment and with only laser irradiation was used as a control group. A sample with RCP irradiation for 10 minutes, a sample with LCP irradiation for 10 minutes, and a sample with RCP irradiation for 5 minutes followed by LCP irradiation for 5 minutes were prepared as control groups to conduct a test. A sample treated with only D-Au NP@PEG was subjected to RCP irradiation for 10 minutes, a sample treated with only L-Au NP@Pd@PEG was subjected to LCP irradiation for 10 minutes, and a sample treated with both D-Au NP@PEG and L-Au NP@Pd@PEG was subjected to RCP irradiation for 5 minutes and LCP irradiation for 5 minutes in sequence. Then, Ez-cytox was diluted tenfold with DMEM and 100 μL of the diluted solution was added to each well and incubated for 30 minutes. Thereafter, absorbance at 450 nm was measured by using a multi-reader.

### 2) Result

Light toxicity in the presence of laser irradiation is shown in the graph of FIG. 15. Referring to FIG. 15, it can be seen that no toxicity was observed from the control sample and the sample with only laser irradiation, whereas toxicity was observed from the samples treated with both the materials and the laser. Particularly, it can be seen that the sample treated with both D-Au NP@PEG and L-Au NP@Pd@PEG exhibits greater toxicity than the sample treated with only D-Au NP and the sample treated with only L-Au NP@Pd. This confirms that hydroxyl radicals are produced through consecutive enzymatic reactions of D-Au NP@PEG and L-Au NP@Pd@PEG and effectively kill cancer cells.

In conclusion, it was confirmed from Test Examples 1 to 4 that D-Au NP oxidizes D-glucose through glucose oxidase-like activity to produce hydrogen peroxide and the produced hydrogen peroxide becomes a substrate for palladium-introduced L-Au NP (L-Au NP@Pd), which finally produces hydroxyl radicals through peroxidase-like activity of palladium and thus exhibits photodynamic therapy to kill cancer cells.

It would be understood by a person with ordinary skill in the art that various changes and modifications may be made based on the above description without changing technical conception and essential features of the present disclosure. Thus, it is clear that the embodiments are illustrative in all aspects and do not limit the present disclosure. The scope of the present disclosure is defined by the following claims. It shall be understood that all modifications and embodiments conceived from the meaning and scope of the claims and their equivalents are included in the scope of the present disclosure.

The scope of the present disclosure is defined by the following claims rather than by the detailed description of the embodiment. It shall be understood that all modifications and embodiments conceived from the meaning and scope of the claims and their equivalents are included in the scope of the present disclosure.

1. A pharmaceutical composition, comprising:
a first nanozyme pharmaceutical composition including a first nanozyme including a first metal nanostructure having a chiral structure; and
a second nanozyme pharmaceutical composition including a second nanozyme including a second metal nanostructure having a chiral structure and a metal coating layer,
wherein each of the enzymatic activities of the first nanozyme and the second nanozyme is improved when circularly polarized light is irradiated thereto, and
the pharmaceutical composition is used for preventing or treating proliferative diseases.

2. The pharmaceutical composition of claim 1,
wherein the first metal nanostructure includes at least one selected from Au, Pd, Pt, Ce, Mn, Ir, and Rh.

3. The pharmaceutical composition of claim 1,
wherein the first nanozyme exhibits glucose oxidase-like activity.

4. The pharmaceutical composition of claim 1,
wherein the first nanozyme has D-directionality.

5. The pharmaceutical composition of claim 1,
wherein the second metal nanostructure includes at least one selected from Au, Pd, Ce, Fe, Cu, Co, and Ir.

6. The pharmaceutical composition of claim 1,
wherein the metal coating layer includes at least one selected from Au, Pd, Pt, Ce, Fe, Cu, Mn, Mo, Co, Ir, Ru, and Ag.

7. The pharmaceutical composition of claim 1,
wherein the second nanozyme exhibits peroxidase-like activity.

8. The pharmaceutical composition of claim 1,
wherein the second nanozyme has L-directionality.

9. A method of preventing or treating proliferative diseases, comprising:
a process of injecting a pharmaceutical composition of claim 1 into non-human subjects; and
a process of irradiating right-handed circularly polarized light and left-handed circularly polarized light in sequence to the site injected with the pharmaceutical composition.

10. The method of claim 9,
wherein hydroxy radicals are produced from D-glucose by the method.

11. The method of claim 9,
wherein the method has a photothermal therapy effect and/or a photodynamic therapy effect.

12. The method of claim 9,
wherein the proliferative diseases include at least one selected from solid tumor, blood cancer, colorectal cancer, uterine cancer, uterine fibroid, meningioma, lung cancer, small cell lung cancer, gastrointestinal cancer, colon cancer, intestinal cancer, breast cancer, ovarian cancer, prostate cancer, testicular cancer, liver cancer, kidney cancer, bladder cancer, pancreatic cancer, brain cancer, sarcoma, osteosarcoma, Kaposi's sarcoma, and melanoma.
